(19) United States
(12) Patent Application Publication
(10) Pub. No.: US 2019/0255204 A1
(43) Pub. Date: Aug. 22, 2019

(54) DECONTAMINATION SYSTEM USING FORCED AIR AND METHODS OF USING THE SAME

(71) Applicant: Medivators Inc., Minneapolis, MN (US)

(72) Inventor: Michael Petersen, Eden Prairie, MN (US)

(21) Appl. No.: 16/343,810

(22) PCT Filed: Nov. 2, 2017
(86) PCT No.: PCT/US17/59670

§ 371 Date: Apr. 22, 2019

Related U.S. Application Data
(60) Provisional application No. 62/418,512, filed on Nov. 7, 2016.

(51) Int. Cl. A61L 2/22; A61L 2/20; A61L 2/26

(52) U.S. Cl. CPC A61L 2/22; A61L 2/20; A61L 2/26; A61L 2202/24; A61L 2202/14; A61L 2202/15; A61L 2/208

(57) ABSTRACT

The invention is directed towards a system and method for decontaminating a device. The system comprises a chamber defining an enclosed space and includes a pump. The enclosed space is configured to withstand pressure changes within the space and receive a device that includes a lumen. The system includes a controller configured to control a temperature and pressure within the chamber, a vacuum pump in fluid communication with the enclosed space, and a source of a decontaminating substance. A vaporizer or atomizer is in fluid communication with the source of the decontaminating substance and is configured to provide a vaporized decontaminating substance within the enclosed space. The pump has an inlet and an outlet. One of the inlet and outlet is in fluid communication with the enclosed space of the chamber and the other of the inlet and outlet is configured to be in fluid communication with the lumen.

### CROSS REFERENCE TO RELATED APPLICATIONS

This application claims priority to U.S. Provisional Patent Application Ser. No. 62/418,512 filed Nov. 7, 2016. This application is incorporated herein by reference, in its entirety.

### TECHNICAL FIELD

The present disclosure relates to decontamination of devices, such as medical devices. More particularly, the present disclosure relates to systems and methods for decontaminating medical devices having a lumen.

### BACKGROUND

Advanced medical instruments formed of rubber and plastic components with adhesives are delicate and often unsuited to the high temperatures and pressures associated with a conventional steam autoclave. Steam autoclaves often operate under pressure cycling programs to increase the rate of steam penetration into the medical devices or associated packages of medical devices undergoing sterilization. Steam sterilization using gravity, high pressure, or pre-vacuum, creates an environment where rapid changes in temperature or pressure can take place. Complex instruments which are often formed and assembled with very precise dimensions, close assembly tolerances, and sensitive optical components, such as endoscopes, may be destroyed or have their useful lives severely curtailed by harsh sterilization methods employing high temperatures and high or low pressures.

Endoscopes can present certain problems in that such devices typically have numerous exterior crevices and interior lumens which can harbor microbes. Microbes can be found on surfaces in such crevices and interior lumens as well as on exterior surfaces of the endoscope. Other medical or dental instruments which comprise lumens, crevices, and the like can also provide challenges for decontaminating various internal and external surfaces that can harbor microbes. There is thus a need for a decontamination system or process that can be used to adequately decontaminate a device having a lumen without risking damage to the device.

### SUMMARY

Disclosed herein is a system for decontaminating a device. The system comprises a chamber defining an enclosed space. The chamber is configured to withstand pressure changes within the enclosed space and receive a device that includes a lumen. The system further includes a controller configured to control a temperature and pressure within the chamber, a vacuum pump in fluid communication with the enclosed space, and a source of a decontaminating substance. The system further includes at least one of a vaporizer or atomizer in fluid communication with the source of decontaminating substance and configured to provide a vaporized decontaminating substance within the enclosed space, and a pump positioned within the chamber. The pump has an inlet and an outlet wherein one of the inlet and outlet is in fluid communication with the enclosed space of the chamber and the other of the inlet and the outlet is configured to be in fluid communication with the lumen.

Also disclosed herein is a system for decontaminating a device having a lumen. The system comprises a decontamination chamber defining an enclosed space. The decontamination chamber is configured to withstand pressure changes within the enclosed space. The system further includes a container positioned within the enclosed space. The container has an outer wall that is permeable by a vaporized decontaminating substance. The system further includes a device having a lumen, the device may be positioned within the container; a controller for controlling a temperature and pressure within the decontamination chamber; a vacuum pump in fluid communication with the enclosed space; and a source of a decontaminating substance. The system further includes at least one of a vaporizer or atomizer for vaporizing the decontaminating substance, the vaporizer or atomizer in fluid communication with the source of the decontaminating substance and the enclosed space. The system further includes a pump positioned within the decontamination chamber, the pump having a first opening in fluid communication with the enclosed space and a second opening in fluid communication with the lumen.

Also disclosed herein is a method of decontaminating a device containing a lumen. The method comprises placing the device inside a container and placing the container within a decontamination chamber defining an enclosed space. The method further includes decreasing the pressure inside the enclosed space to a pressure below atmospheric pressure, directing a vaporized decontaminating substance into the enclosed space, and providing a flow of air containing the vaporized or atomized decontaminating substance from the enclosed space into a lumen of the device with a pump located within the enclosed space or the container.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of a decontamination system according to some embodiments.

FIG. 2 is a graph showing pressure versus time in an exemplary decontamination cycle.

FIG. 3 is a schematic view of a decontamination system according to some embodiments.

FIG. 4 is a schematic view of a decontamination system according to some embodiments.

FIG. 5 is a schematic view of a decontamination system according to some embodiments.

FIG. 6 is a schematic view of a decontamination system according to some embodiments.

FIG. 7 is a schematic view of a decontamination system for a multilumen device.

FIG. 8 is a schematic view of an alternative decontamination system for a multilumen device.

### DETAILED DESCRIPTION

Devices, such as medical devices, can be decontaminated or sterilized at relatively low temperatures using hydrogen peroxide (H₂O₂) and/or peracetic acid (PAA) chemistry. In such systems, the chemistry may be provided as a vapor into a decontamination chamber containing the device to be decontaminated. The surfaces of the device will he decontaminated when contacted with the chemistry. Lumen devices may be particularly challenging to decontaminate as there must be flow of the decontaminating substance through the lumen. The instant disclosure describes a decontamination system which provides sufficient flow of the decontaminating substance through a lumen device to achieve decontamination of the lumen device. A method of using is also described.

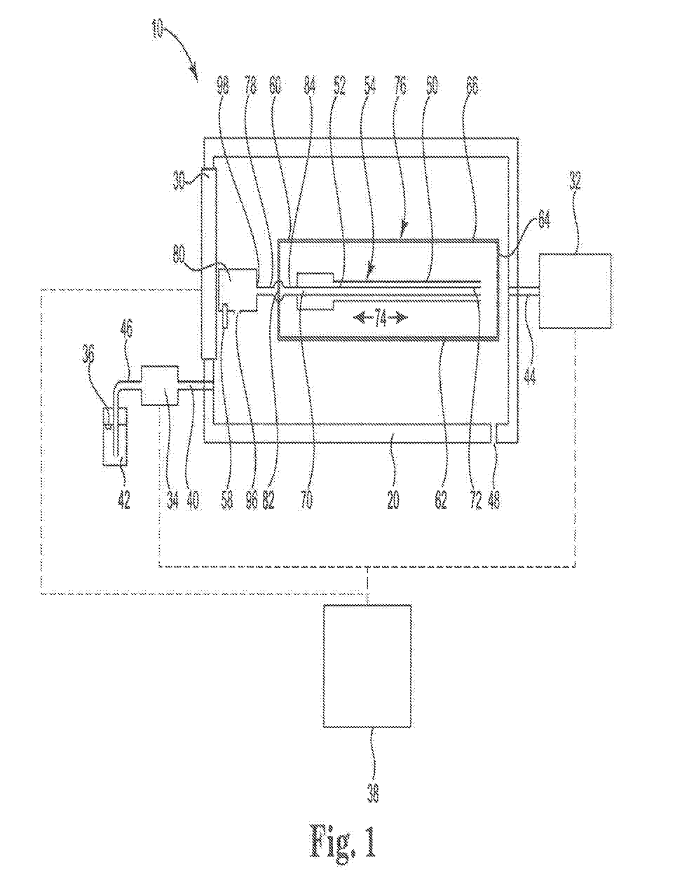

FIG. 1 is a schematic view of a decontamination system 10 which includes a decontamination chamber 20, a vacuum pump 32, a vaporizer 34, a source of decontaminating substance 36 maintained in a package 42, a controller 38, a vent 48, a pump 80, and a device 50 positioned within a container 60. Vacuum pump 32 is connected to decontamination chamber 20 by conduit 44. Vaporizer 34 is connected to decontamination chamber 20 by conduit 40. Decontaminating substance 36 is maintained in package 42 and is connected to vaporizer 34 by conduit 46. Controller 38 is connected to the vacuum pump 32, vaporizer 34, and pump 80 and is configured to control these items. In some embodiments, controller 38 is connected to one or more of the decontamination chamber 20, vacuum pump 32, vaporizer 34, packaging 42 and pump 80.

Decontamination chamber 20 defines an enclosed space. The decontamination chamber 20 includes a door 30 that is configured to accommodate inserting or removing the container 60. The decontamination chamber 20 is configured to withstand pressure changes within the decontamination chamber 20 as described herein. The door 30 may be sealed and/or reinforced to provide a sealed environment within the decontamination chamber 20 that is higher or lower than the pressure outside the decontamination chamber 20.

The vacuum pump 32 is connected to the decontamination chamber 20 and is configured to change the pressure within the decontamination chamber 20. For example, the vacuum pump 32 may be configured to withdraw gas from the decontamination chamber 20 to lower the pressure inside the decontamination chamber 20. The vacuum pump 32 may also be configured to add gas into the decontamination chamber 20 to increase the pressure inside the decontamination chamber 20.

In some embodiments, the decontamination chamber 20 may also include the vent 48 to adjust the pressure within the decontamination chamber. For example, the vent 48 may be controlled by controller 38 to be in a partially or fully open position or in a closed position. In some embodiments, the vent 48 may be in a closed position when the vacuum pump 32 is adjusting the pressure inside the decontamination chamber 20 above or below atmospheric pressure and may be in a partially or fully open position when the vacuum pump 32 is adjusting the pressure inside the decontamination chamber 20 atmospheric pressure. For example, the vent 48 may be in a partially or fully open position such that atmospheric air can enter the decontamination chamber 20 when the vacuum pump 32 is drawing air from the decontamination chamber 20 to flush air through the decontamination chamber 20.

The package 42 containing the decontaminating substance 36 and the vaporizer 34 are located outside the decontamination chamber 20. The package 42 is connected to the vaporizer 34 by conduit 46 and the vaporizer 34 is connected to the decontamination chamber 20 by conduit 40. Together conduits 46 and 40 provide a fluid connection from the package 42 into the decontamination chamber 20 such that the decontaminating substance 36 flows from the packaging 42 through the vaporizer 34 into the decontamination chamber 20.

The decontaminating substance 36 may include chemistry suitable for use in a sterilization process. For example, the decontaminating substance 36 may include a chemical or other substance that complies with the International Organization for Standardization (ISO) standard. ISO/TC 198, Sterilization of Healthcare Products and/or the Association for the Advancement of Medical Instrumentation (AAMI) standard ANSI/AAMI/ISO 11140-1:2005, “Sterilization of Healthcare Products—Chemical Indicators—Part I: General Requirements” (Arlington, Va.: AAMI 2005). In some embodiments, suitable decontaminating substance 36 includes chemistry that can be dispersed as a fluid, such as a liquid, a vapor, or a combination thereof (such as a fog) during the decontamination process. For example, suitable decontamination substances may include hydrogen peroxide (H₂O₂) and/or peracetic acid (PAA). Decontaminating substance 36 may be kept at room temperature (e.g., 20° C. to 30° C.) before being provided to the vaporizer 34. Decontaminating substance 36 may be refrigerated or heated before being provided to the vaporizer 34.

The vaporizer 34 converts the decontaminating substance 36 into a vapor, fog or other suitable form for the decontamination process. For example, in some embodiments the vaporizer 34 may beat the decontaminating substance 36 provided in a liquid form to vaporize or otherwise transform the liquid decontaminating substance 36 into a vapor or gas. In some embodiments, the vaporizer 34 may convert the decontaminating substance 36 into a vapor or fog via a mechanical means such as an atomizing nozzle or a sprayer. For example, the vaporizer may include an atomizer that uses a mechanical force such as rotating blades or air pressure to break up a stream of liquid decontaminating substance 36 into individual droplets and/or to produce an aerosol. The droplets or aerosol of decontaminating substance 36 may be released into the decontamination chamber 20 where they may evaporate into the gas phase to form a vapor. In some embodiments, the decontaminating substance 36 may be pulled into the vaporizer 34. In other embodiments, the decontaminating substance 36 may be pushed into the vaporizer 34.

The controller 38 provides control signals to and/or receives condition sensing and equipment status signals from other elements of the decontamination system 10. For example the controller 38 may include monitoring and control of the vaporizer 34, the vacuum pump 32, and the pump 80. The controller 38 may regulate delivery of the decontaminating substance 36 to the vaporizer 34. The controller 38 may adjust the environmental conditions within the decontamination chamber 20. The controller 38 may provide control signals to and/or receive condition sensing and equipment status signals from the vacuum pump 32 for adjustment of the pressure of the decontamination chamber 20.

The decontamination chamber 20 is configured to receive the device 50 within a container 60. The device 50 may include one or more lumens 52 having a first end 70, a second end 72 and a length 74. The lumen 52 extends the length of the device 50, and has an inner diameter that is smaller than the length 74 of the lumen. For example, the device 50 may be a medical device, such as an endoscope, having a rigid or flexible lumen that extends the length of the device. In some embodiments, an endoscope lumen may have a length several times larger than the inner diameter. For example, an endoscope lumen may have an inner diameter of about 1 to 4 millimeters and may have a length of about 1 to 3 meters (m).

In some embodiments, the decontamination chamber 20 may be operated at room temperature (e.g., 20° C. to 30° C.), below room temperature, or above room temperature. For example, in some embodiments, the decontamination chamber 20 may include a heater or other device for increasing the temperature of the decontamination chamber 20. In some embodiments, the decontaminating substance 36 can be heated to a vapor and the vapor may heat the decontamination chamber 20 to a temperature above room temperature (e.g., above 20° C. or 30° C.).

The container 60 forms an enclosed space and holds one or more devices 50. The container 60 may have one or more sides or portions 64 that forms the enclosed space. Sides or portions 64 may be flexible or rigid. The sides or portions 64 of container 60 may be of the same material or different materials. For example, the container 60 may be a flexible pouch made entirely or partially from one or more pliable or flexible portions 64. In another example, the container 60 may be a case or other enclosure formed from a rigid material. In a further example, container 60 may have a rigid bottom 62 and portions 64 and may have a flexible top 66 or lid. In some embodiments, the container 60 may be disposable. In other embodiments, the container 60 may be reusable. The container 60 may be designed to contain the device 50 during a decontamination process, and maintain the device 50 in a sterile condition after the device 50 is removed from the decontamination chamber 20.

The container 60 may have at least a section of a surface 76 through which the decontaminating substance 36 may penetrate or permeate. For example, vaporized decontaminating substance 36 in the decontamination chamber 20 may contact and decontaminate the outer surface 76 of the container 60. The vaporized decontaminating substance 36 in the decontamination chamber 20 may also permeate through the outer surface 76 of the container 60, enter the container 60, and decontaminate at least the outer surface 54 of the device 50.

The pump 80 is connected to the lumen 52 of the device 50 and is positioned within the decontamination chamber 20 and outside of the container 60. In some embodiments, the pump 80 may be attached to an inside surface of the decontamination chamber 20. The pump 80 is configured to provide a sufficient flow rate so that the fluid from decontamination chamber 20 flows the entire length 74 of the lumen 52.The pump 80 may be any suitable device for moving air or for inducing material flow. For example, the pump 80 may be an air pump, a fan, a compressor, a blower, or bellows

The pump 80 has an inlet 96 and an outlet 98. Fluid enters the pump 80 through the inlet 96 and exits the pump 80 through the outlet 98. The inlet 96 is exposed to, or is in fluid communication with, the environment within the decontamination chamber 20. In some embodiments, the decontamination system 10 provides fluid communication from the pump 80 to the device 50 to direct air flow from the pump to the device 50 and the lumen 52. For example, during a decontamination cycle, the pump 80 directs air containing decontaminating substance 36 into the lumen 52 of the device 50. In some embodiments, the outlet 98 may be spaced a distance from the lumen 52 and direct a portion of the air flow around the lumen 52 and a portion of the air flow through the lumen 52

In some embodiments, the pump 80 may be powered using a magnetic coupling. For example, the pump 80 may be positioned on the inside surface of a wall of the decontamination chamber 20 and may be powered using a magnetic coupling to turn the pump 80 using a power source or mechanical force driving the pump 80 from outside the decontamination chamber 20. Additionally or alternatively, the pump 80 may be battery powered. In some embodiments, the pump 80 may be configured to turn on while door 30 is open. Additionally or alternatively, the pump 80 may be configured to turn on when the door 30 is in the closed position. For example, controller 38 may turn on pump 80 when the door 30 is in the closed position.

In some embodiments, the system 10 may include a sensor 58 for detecting the air pressure inside the decontamination chamber 20. When a suitable air pressure inside the decontamination chamber 20 is detected, the pump 80 may be turned on by controller 38, for example.

In some embodiments, the pump 80 is in fluid communication with device 50 and pump 80 forces air into lumen 52 of device 50. In some embodiments, the pump 80 may be in fluid communication with the device 50 via a conduit 78 that is attached to the pump outlet 98. The conduit 78 may be used to direct a flow of air from the pump 80 to any suitable location such as the device 50 and/or the lumen 52. The conduit 78 may be attached to the device 50 directly or via an additional connection. For example, the conduit 78 may be attached to a port 82 that provides fluid communication from the conduit 78 to the device 50 inside the container 60. In some embodiments, the device 50 may not he contained within a container 60, and the device 50 may be directly connected to the outlet 98 of the pump 80 or may be connected to the conduit 78 which is connected to the outlet 98 of the pump 80.

In some embodiments, the device 50 may be in fluid communication with the outlet 98 of the pump 80 through a direct connection or through a series of intermediate connections. For example, the port 82 may be positioned on the container 60 to direct flow from the pump 80 through a side 64 or top 66 of the container 60 to lumen 52. The port 82 may include a portion extending from the outside of the container 60 and a portion extending into the container 60. The port 82 may be used to connect the device 50 in fluid communication with the pump 80. In some embodiments, conduit 78 connects the pump 80 to port 82, and the lumen 52 may be connected to the port 82. In some embodiments, conduit 78 connects the pump 80 to port 82, and a conduit 84 connects port 82 to the lumen 52. The port 82 may include a permeable material across the port 82 that allows vaporized decontaminating substance 36 to permeate through. Suitable permeable materials include, for example, a nonwoven material such as that sold under the tradename Tyvek®. The permeable material across the port 82 allows a sterile environment to be maintained within the container 60 after the container 60 is removed from chamber 20. The port 82 may be attached to conduit 78 using any suitable connection such as a threaded connection, a snap connection that clamps around the connection, or a quick connect that has a male internal pipe that may be inserted into a spring loaded female external pipe that may be removed by retracting the female pipe which releases the male connection. In some embodiments, pump 80 may be directly connected to wall 92 without a port 82.

As shown in FIG. 1, the pump 80 is connected to the device 50 at the first end 70 of the lumen 52. In use, the pump 80 may push air that contains vaporized decontaminating substance 36 into the first end 70 of the lumen 52. The pump 80 may also pull air from the first end 70 of the lumen 52 which causes air containing vaporized decontaminating substance 36 to be drawn into the second end 72 of the lumen 52. Air in the decontamination chamber 20 that contains vaporized decontaminating substance 36 may permeate through the container 60 and be drawn into the second end 72 of the lumen 52. In some embodiments, the pump 80 may be designed to alternate between pushing and pulling air from inside the lumen 52. In this way, the pump 80 can provide flow in either direction along the length 74 of the lumen 52.

In some applications, it is challenging to achieve adequate decontamination of endoscopes due to the long, narrow lumens. Material transfer along the inside of the length of an endoscope is often difficult to achieve with passive diffusion of the chemistry. For example, an endoscope may have a lumen 52 with a length 74 of 1.0 meters, 2.0 meters, 3.0 meters or greater, and may have an internal diameter of 0.2 mm, 0.5 mm, 1.0 mm, 2.0 mm, 3.0 mm, or 4.0 mm. Systems utilizing passive diffusion of the chemistry down the length of the endoscope typically require long contact times and/or high chemistry concentrations to achieve adequate decontamination. Long processing times and/or high concentrations increase the risk of damaging the endoscope. Long processing times also lead to operating higher costs, as fewer devices can be decontaminated in a given amount of time.

During a decontamination process, the pump 80 threes decontaminating substance 36 through lumen 52 and along the entire length 74 of the lumen 52. For example, pump 80 forces or directs air or other fluid containing decontaminating substance 36 into the lumen 52 at a suitable volumetric flow rate to travel the length 74 of the lumen 52. In this manner, the entire surface of the lumen 52 is contacted with decontaminating substance 36.

In some embodiments, the pump 80 may provide a suitable volumetric flow rate to create a turbulent flow along the interior of the lumen 52. In other embodiments, the pump 80 may produce a laminar flow along the interior of lumen 52. The flow rate of the pump 80 may be adjusted based on the interior diameter of the lumen 52 to provide the suitable type of flow (i.e., laminar or turbulent).

The volumetric flow rate provided by the pump 80 may be predetermined to provide a specified amount of decontaminating substance 36 at a particular rate. For example, the concentration of vaporized decontaminating substance 36 in the air in the decontamination chamber 20 may be calculated, and the required volume of air that contains a suitable amount of decontaminating substance 36 may be forced through the lumen 52 in a suitable amount of time to achieve a required decontamination.

The decontamination system 10 with the pump 80 allows a user to reduce the risk of damage to the device to be decontaminated by reducing the time and the chemistry concentration needed to achieve sufficient or suitable decontamination. A faster decontamination process also allows a shorter decontamination process to be used, which increases the number of devices that can be processed with a single decontamination system 10 during a given time period. Additionally, a lower concentration of the decontaminating substance 36 provides a decontaminating process that requires less decontaminating substance 36 and less time to operate the decontamination system 10, thus decreasing the cost of the decontamination process.

The decontamination process is described below with reference to the decontamination system 10 described in FIG. 1, and the corresponding reference numbers. However, the decontamination process described herein may be carried out by any of the decontamination systems 10, 210, 310, 410 shown in FIGS. 1, 3, 4, 5, and 6. As described herein, a decontamination process includes at least one decontamination cycle, in which a decontamination cycle includes at least one release of decontaminating substance 36 into the decontamination chamber 20. In some embodiments, a decontamination process may include two or more identical decontamination cycles. The first step of a decontamination cycle may be decreasing the pressure within the decontamination chamber 20 below atmospheric pressure, and the last step may be returning the pressure within the decontamination chamber 20 to atmospheric pressure. In some embodiments, a decontamination process begins when the device 50 is placed within the decontamination chamber 20, and ends when the device 50 is removed from the decontamination chamber 20.

In some embodiments, to decontaminate the device 50 and lumen 52, the device 50 may be placed within the container 60 and the lumen 52 is attached to the conduit 84. The device 50 may be sealed within the container 60 and placed in the decontamination chamber 20. The container 60 may be connected to the pump 80 by connecting the conduit 84 to the port 82.

After the device 50 is placed within decontamination chamber 20, the door 30 is closed and sealed. The pressure within the decontamination chamber 20 may then be decrease to below atmospheric pressure and decontaminating substance 36 may be introduced into the decontamination chamber 20. In some embodiments, the pressure may be decreased at the same time that decontaminating substance 36 is introduced. Alternatively, decontaminating substance 36 may be introduced after the pressure is reduced in the chamber 20.

In some embodiments, the decontaminating substance 36 is introduced into the decontamination chamber 20 after being converted to a vapor or fog. For example, a vapor may be generated by delivering decontaminating substance 36 into the vaporizer 34 where the decontaminating substance 36 is vaporized. The vaporized decontaminating substance 36 is then introduced into the decontamination chamber 20, under sub-ambient pressure where it tills the decontamination chamber 20. The decontaminating substance 36 may be introduced into the decontamination chamber 20 when the pressure of the decontamination chamber 20 is lower than atmospheric pressure, for example less than about 100 Torr, less than about 50 Torr, or less than about 10 Torr.

In some embodiments, the decontaminating substance 36 may contain about 59% hydrogen peroxide, and the balance water. In other embodiments, the decontaminating substance 36 may contain peracetic acid (PAA). For example, the decontaminating substance 36 may include a chemistry that delivers PAA at a suitable concentration into the decontamination chamber 20. In some embodiments, suitable concentrations may include a weight percentage of PAA by weight of the decontaminating substance 36 from about 1.0 wt. % to about 8.0 wt. %, from about 2.0 wt. % to about 7.0 wt. %, or from about 3.0 wt. % to about 6.0 wt,%, or any weight percentage within these ranges.

In some embodiments, decontaminating substance 36 is provided in a premeasured volume sufficient to decontaminate the device 50 and the lumen 52. For example, in some embodiments, a decontamination process includes transferring a predetermined amount of a decontaminating substance 36, such as aqueous hydrogen peroxide or peracetic acid (PAA), to the package 42. Additionally or alternatively, the decontaminating substance 36 may be provided in a large or bulk amount and decontamination system 10 may provide a sufficient amount of decontaminating substance 36 to vaporizer 34 for a particular decontamination cycle.

As described herein, at least a section of the outer surface 76 of the container 60 containing device 50 is permeable to the vaporized decontaminating substance 36. During a decontamination cycle, the vaporized decontaminating substance 36 will permeate through the outer surface 76 of the container 60 and decontaminate the outer surface 54 of the device 50. The pump 80 forces air containing vaporized decontaminating substance 36 from decontamination chamber 20 through the lumen 52 by either pushing or pulling the air. Forcing air through the lumen 52 increases the flow rate of air containing decontaminating substance 36 through the lumen 52. This increases the amount of chemistry to which the lumen 52 is exposed and decreases the cycle time and/or chemistry concentration required to achieve decontamination of the device 50 and the lumen 52.

The decontaminating substance 36 may be held in decontamination chamber 20, and/or in lumen 52, for a period of time to facilitate decontamination of the device 50. When the decontaminating substance 36 has been held for a suitable amount of time, the controller 38 can vent the decontamination chamber 20 to a higher, but in some embodiments, still sub-atmospheric pressure. An air wash may be used to remove the vapor containing the decontaminating substance 36 from the decontamination chamber 20 and device 50. During the air wash, the controller 38 may increase the pressure within the decontamination chamber 20 and then decrease the pressure within the decontamination chamber 20. In some embodiments, the pump 80 may direct air into the device 50 to remove any residual decontaminating substance 36 in lumen 52. After the air wash, the inside of the decontamination chamber 20 can he returned to atmospheric pressure. While multiple embodiments for providing fluid flow through device 50, and specifically through lumen 52, are envisioned, in each embodiment, it is an object of the instant disclosure to provide sufficient decontaminating substance 36 along the length of the lumen 52 to decontaminate the lumen 52. In various embodiments, the instant application discloses a system that enables forcing air containing vaporized decontaminating substance 36 down the length of the lumen 52.

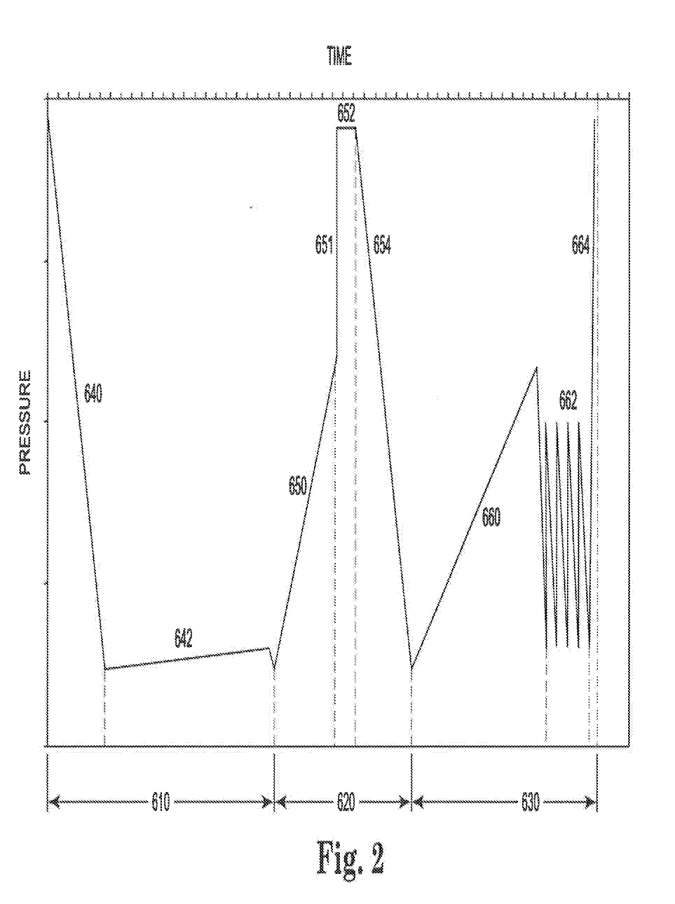

FIG. 2 shows a graph of pressure versus time within decontamination chamber 20 in an example embodiment of a decontamination cycle. As shown in FIG. 2, the X-axis of the graph illustrates time or duration, and the Y-Axis illustrates pressure within the decontamination chamber. As shown in FIG. 2, in some embodiments, a decontamination cycle may include multiple pressure changes within the decontamination chamber. The decontamination cycle or a portion of the decontamination cycle illustrated in FIG. 2 may be repeated several times within a decontamination process.

The decontamination cycle of FIG. 2 includes a vacuum preconditioning step 610, a first decontamination step 620, and a second decontamination step 630. The vacuum preconditioning step 610 includes a first pump down 640 in which pressure is drawn from the decontamination chamber and an optional lumen warm up period 642. During the lumen warm up period 642, the pressure within the decontamination chamber is held relatively steady.

In some embodiments, the vacuum preconditioning step 610 may be followed by the first decontamination step 620. During the first decontamination step 620, decontaminating substance is injected into the decontamination chamber in a first injection step 650. During the first injection step 650 the pressure within the decontamination chamber increases. In an example embodiment, of decontaminating substance is injected into the decontamination chamber during the first injection step 650. The decontaminating substance may be injected into the decontamination chamber at a single injection at a constant rate as shown in the first injection step 650 or it may be injected in a plurality of stepwise injections.

The first injection step 650 may be optionally followed by a pressure increase step 651. During the pressure increase step 651, the pressure inside the decontamination chamber is increased to a suitable pressure determined to increase the effectiveness of a decontamination process. After the decontaminating substance is injected, it may be optionally allowed to diffuse throughout the decontamination chamber in a diffusion period 652 while the pressure is held steady. In some embodiments, the optional diffusion period 652 is not used.

In some embodiments, after the diffusion period 652, a second pump down 654 may be carried out. During the second pump down 654, the pressure within the decontamination chamber decreases. The second decontamination step 630 is carried out after the second pump down 654. During the second decontamination step 630, a second injection step 660 may be used to add decontaminating substance to the decontamination chamber while the pressure within the decontamination chamber increases. The second injection step 660 may include adding decontaminating substance into the decontamination chamber in a single injection step or in a plurality of stepwise injection steps that may be used to gradually add decontaminating substance to the decontamination chamber.

In some embodiments, a pump may be used to direct air within the decontamination chamber through the lumen or lumens of the device in coordination with the decontamination cycle. For example, during the first injection step 650, the second injection step 660 or both injection steps, a pump may be used to direct air within the decontamination chamber towards and/or through the lumens of the device. In some embodiments, the pump may be turned on before or during either the first or second injection step 650, 660. For example, the pump may be turned on with or substantially with the first and/or second injection steps 650, 660. In some embodiments, the pump may turn on before or during the first injection step 650 and may turn off at the end of or after the first injection step 650. Additionally or alternatively, the pump may turn on before or during the second injection step 660 and may turn off after or at the end of the second injection step 660. In some embodiments, the pump may turn on before or during both the first and second injection steps 650, 660, or the pump may be turned on before or at the beginning of the first injection step 650 and may be turned off during or after the end of the second injection step 660.

After the second injection step 660, a plurality of air washes 662 may be carried out. As shown in FIG. 2, the plurality of air washes 662 may include increasing and decreasing the pressure within the decontamination chamber repeatedly. In some embodiments, the pump 80 may be run during the plurality of air washes 662 to force air along the inside of the device to be decontaminated. The air washes may be carried any number of times to remove a suitable amount of decontaminating substance from the decontamination chamber. After a suitable number of air washes 662, the pressure within the decontamination chamber may be allowed to reach atmospheric pressure in a final vent step 664.

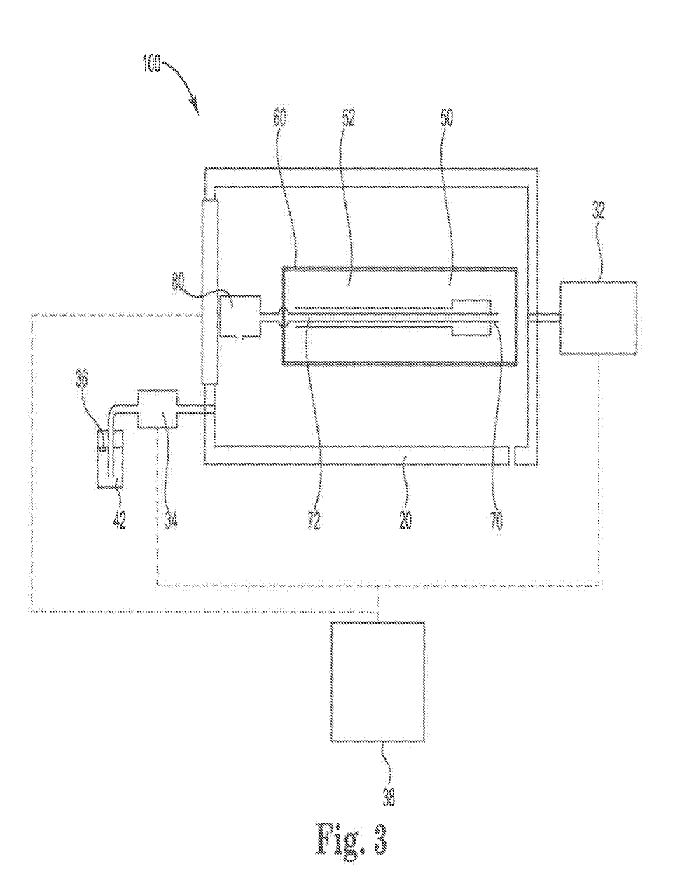

FIG. 3 is a schematic view of the decontamination system 100 which includes a decontamination chamber 20, a vacuum pump 32, a vaporizer 34, a source of decontaminating substance 36 maintained in a package 42, a controller 38, a device 50 positioned within a container 60, and a pump 80. As shown in FIG. 3, the pump 80 is connected to the device 50 at the second end 72 of the lumen 52. In this configuration, the pump 80 can pull fluid through the lumen 52 from the first end 70 to the second end 72, or the pump 80 can push air from the second end 72 to the first end 70.

As shown in FIG. 3, the pump 80 may push air that contains vaporized decontaminating substance 36 into the second end 72 of the lumen 52 and the air will exit the lumen 52 at the first end 70. In other embodiments, the pump 80 may be configured to pull air from the second end 72 of the lumen 52 which causes air that contains vaporized decontaminating substance 36 to be drawn into the first end 70 of the lumen 52. Air in the decontamination chamber 20 containing vaporized decontaminating substance 36 may permeate through the container 60 and be drawn into the first end 70 of the lumen 52.

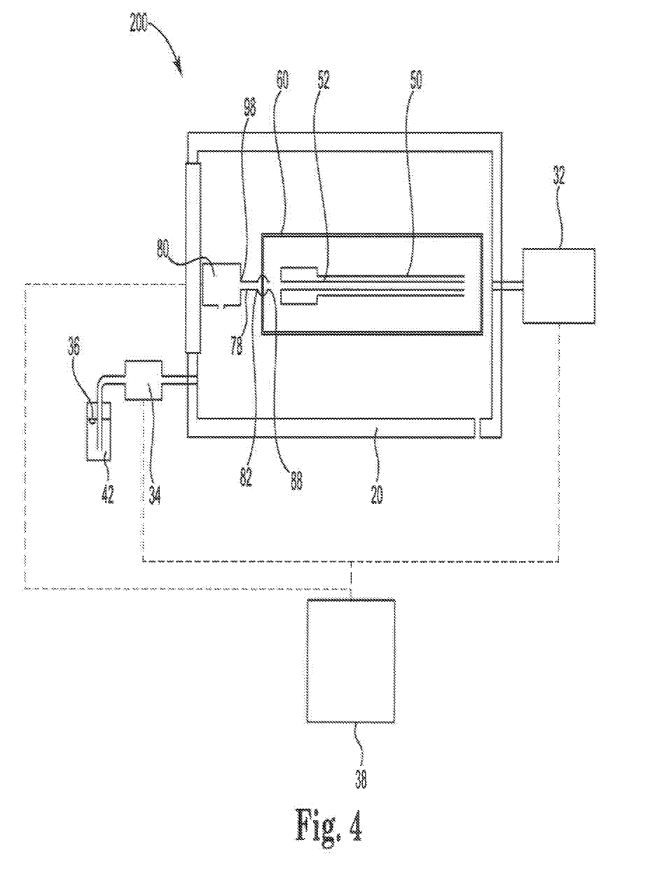

FIG. 4 is a schematic view of a decontamination system 200 which includes a decontamination chamber 20, a vacuum pump 32, a vaporizer 34, a source of decontaminating substance 36 maintained in a package 42, a controller 38, a device 50 positioned within a container 60, and a pump 80. As shown in FIG. 4, in some embodiments, the pump 80 is not directly coupled to the device 50 within the container 60. That is, the structure that is used to provide decontaminating substance 36 to the device 50, such as a conduit 78 that is attached to an outlet 98 of the pump 80 or a port 82 inside the container 60, may terminate at an end 88 that is physically separated, i.e. spaced, from the device 50 and does not touch the device 50. That is, the outlet 98 of the pump 80, the conduit 78, or the port 82 may be physically separated by being spaced a distance from the device 50. As shown in FIG. 4, the structure that is used to direct air into the container 60 such as the port 82 may have an end 88 within the container 60 that is spaced from the device 50. The outlet 98 and/or port 82 may be spaced apart from the device 50 and shaped to direct flow from the pump 80 to the device 50, thus allowing fluid communication between the pump 80 and the lumen 52 without the device 50 touching the pump 80 or any physical connection with the pump 80 such as the outlet 98, the port 82, or the end 88 of the port 82.

In some embodiments, the device 50 may be positioned with suitable spacing between the device 50 and the end 88 of the port 82 or pump outlet 98 to allow a portion of the flow of fluid to flow around the device 50. The spacing may allow a distance or a gap between the end 88 and the device 50 such that the end 88 and the device 50 do not touch while also allowing sufficient flow of fluid through the lumen 52 and a flow of fluid around the device 50. In some embodiments, the first end 54 of the lumen 52 may be spaced about 0.5 cm or less, 1.0 cm of less, or 3.0 cm or less from the end of the fluid flow path from the pump 80, i.e., the end 88 in FIG. 4. In some embodiments, the first end 54 of the lumen 52 and end 88 are not in contact with one another and may be spaced not more than 0.5 cm apart. The first end 54 of the lumen 52 may be spaced from the end 88 of the port 82 or pump outlet 98 at a distance that allows a suitable portion of air leaving the pump 80 to flow through the lumen 52. For example, the first end 54 of the lumen 52 may be spaced from the end 88 of the port 82 to allow at least about 90%, at least about 80%, or at least about 70% of the flow leaving from the end of the fluid flow path from the pump 80 i.e., the end 88 in FIG. 4 to enter and flow through the lumen 52.

In some embodiments, the device 50 may be spaced a suitable distance such that the portion of air flow exiting the port 82 that is directed through the lumen 52 provides a suitable volumetric flow of air through the lumen 52 to achieve sufficient decontamination of the lumen 52 during a decontamination cycle. That is, the portion of air directed through the lumen 52 is a suitable volumetric flow of air such that when multiplied by the amount of time the air is directed through the lumen, a suitable total volume of air is directed through the lumen 52. A suitable volume of air may also be allowed to flow around and decontaminate the outside of the device 50 during a decontamination cycle.

As shown in FIG. 4, in some embodiments, the pump 80 may be configured to force air from within the decontamination chamber 20 into the container 60 and flow both inside and outside the device 50 and the lumen 52. In this configuration, a flow of air from pump 80 containing vaporized decontaminating substance 36 may be channeled toward the lumen 52 of the device 50 with a portion of the flow of air allowed to flow around the outside of the device 50 and a portion of the flow of air allowed to flow through the lumen 52. In some embodiments, the end 88 may be shaped to direct fluid flowing out of the end 88. For example, the end 88 may include a taper or a tip to direct the fluid as it exits the end 88. In some embodiments, the structure that is used to direct air containing decontaminating substance 36 to the lumen 52 may be aligned with the lumen 52. For example, the port 82 may have a central axis that is aligned with a central axis of the lumen 52 and provide a suitable flow of air into the lumen 52 or an inlet of the device 50.

As shown in FIG. 4, air containing decontaminating substance 36 may be channeled or directed to flow through the lumen 52 without having the pump 80 or the outlet 98 of the pump in physical contact with the device 50. In some embodiments, it can be difficult to decontaminate surfaces at connection points, such as at the connection between the device 50 and a conduit from the pump 80. The configuration of system 200 eliminates the connection surface the device 50 and a conduit from pump 80.

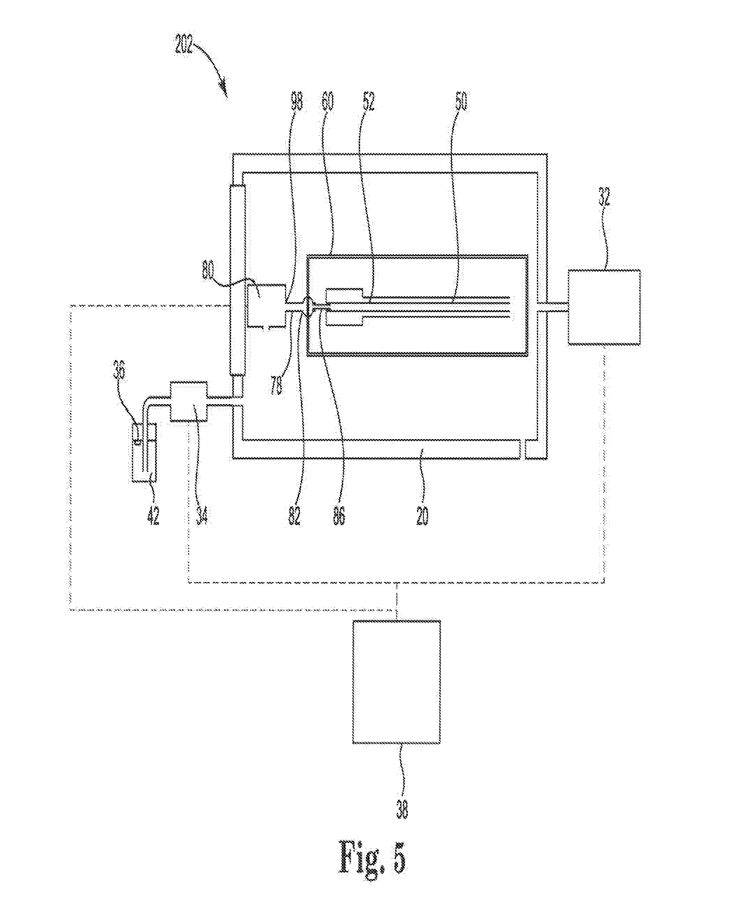

FIG. 5 is a schematic view of a decontamination system 202 which includes a decontamination chamber 20, a vacuum pump 32, a vaporizer 34, a source of decontaminating substance 36 maintained in a package 42, a controller 38, a device 50 positioned within a container 60, and a pump 80. The decontamination system 202 may include structure that is used to provide fluid communication between the pump 80 and the device 50 inside the container 60, such as a conduit 78 that is attached to an outlet 98 of the pump 80 or a port 82. As shown in FIG. 5, an injection attachment 86 is attached to port 82 and is positioned at least partially within the lumen 52 to direct fluid into the lumen 52 without touching the lumen 52. In some embodiments, the injection attachment 86 is sized such that the outer diameter of the injection attachment 86 positioned within the lumen 52 is smaller than the inner diameter of the lumen 52. For example, in some embodiments, the injection attachment 86 fits within the lumen 52 of the device 50 without touching the lumen 52. In system 202, injection attachment 86 can channel air containing vaporized decontaminating substance 36 to the lumen 52 of the device 50 to ensure a suitable flow rate of air is introduced into the lumen 52 for decontamination of the lumen 52 without coming in contact with the lumen 52 and potentially contaminating the lumen 52.

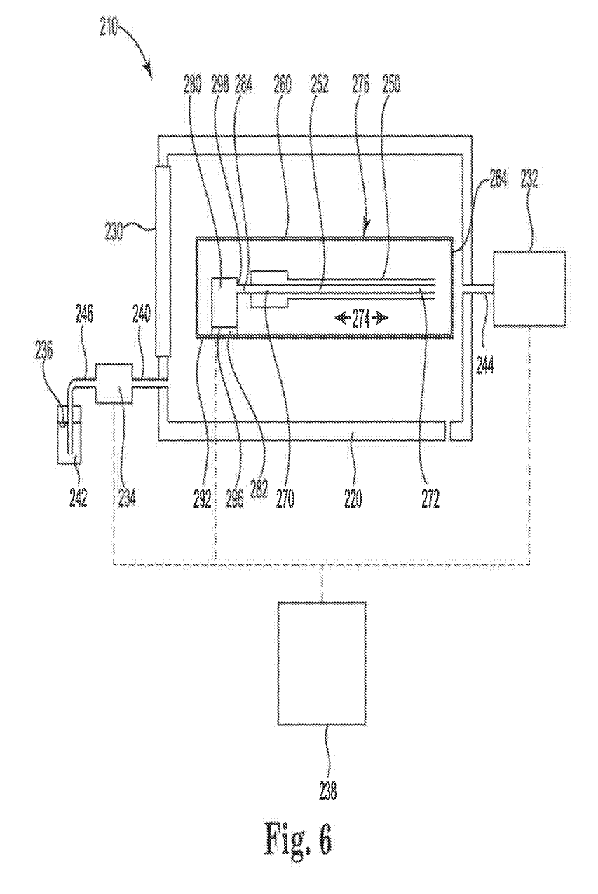

FIG. 6 is a schematic view of a decontamination system 210 which includes a pump 280 inside of a container 260 enclosing a device 250 containing a lumen 252. Container 260 forms an enclosed space that holds the device 250 and the pump 280.

As shown in FIG. 6, decontamination system 210 includes a decontamination chamber 220, a vacuum pump 232, a vaporizer 234, a source of decontaminating substance 236 maintained in a package 242, a controller 238, a device 250 positioned within a container 260, and a pump 280. Vacuum pump 232 is connected to decontamination chamber 220 by conduit 244. Vaporizer 234 is connected to decontamination chamber 220 by conduit 240. Decontaminating substance 236 is maintained in package 242 and is connected to vaporizer 234 by conduit 246. Controller 238 may be connected to and configured to control one or more of the vacuum pump 232, vaporizer 234, and pump 280.

The container 260 may have one or more sides or portions 264 that forms the enclosed space as described above with respect to container 260.

In some embodiments, a port 282 is positioned on the container 260 and connected to the pump inlet 296. The port 282 may be attached to the pump inlet 296 using any suitable connection such as a threaded connection, or a quick connect. The port 282 allows fluid to flow through a wall 292 of the container 260 to the pump inlet 296. That is, the pump inlet 296 is in fluid communication with the decontamination chamber 220 through the port 282. In some embodiments, the pump 280 may be spaced from the wall 292. In some embodiments, a permeable material is positioned across the port 282 that allows vaporized decontaminating substance 236 to permeate through and enables a sterile environment to be maintained within the container 260 after the container 260 is removed from decontamination chamber 220.

In some embodiments, the pump inlet 296 may be directly connected to wall 292 without a port 282. For example, the pump 280 may be attached to the wall 292 on the inside of the container 260 and the pump inlet 296 may be located outside the container 260. In some embodiments, the container 260 does not include a port 282 and the portion of the container aligned with the inlet of the pump 280 is permeable or penetrable by the vaporized decontaminating substance 236. For example, in use, the vaporized decontaminating substance 236 in the decontamination chamber 220 can permeate through the surface 276 of the container 260, enter the container 260, and flows into the pump inlet 296. In some embodiments, the container may have both a port 282 and a section of a surface 276 through which the decontaminating substance 236 may penetrate or permeate. This configuration may allow air from the decontamination chamber 220 to enter through the port 282 and exit through the surface 276 of the container.

The pump 280 is in fluid communication with the lumen 252 and forces or directs air from decontamination chamber 220 into lumen 252 of device 250 during at least a portion of a decontamination cycle. The pump 280 is configured to provide a sufficient flow rate so that the air from decontamination chamber 220 travels an entire length 274 of the lumen 252. The pump 280 may be controlled to provide a suitable volumetric flow rate though the lumen 252 to form turbulent flow along the length 274 of the lumen 252. In this way, pump 280 pushes or forces air from decontamination chamber 220 through lumen 252 along the length 274 to a second end 272 to decontaminate the entire inner surface of the lumen 252.

In some embodiments, the pump outlet 298 is connected to the first end 270 of the lumen 252 by conduit 284, which is similar to system 10 of FIG. 1. In other embodiments, the pump outlet 298 may connected the second of end 272 of the lumen 252, which is similar to system 10 of FIG. 3. In some embodiments, the conduit 284 is not in physical contact with and is spaced apart from the lumen 252, similar to system 200 of FIG. 4. In some embodiments, an injection attachment, similar to that shown in FIG. 5, may be used to direct air into the lumen 252,

The pump 280 may be configured to provide a volumetric flow rate that is suitable for the internal volume of the lumen 252. For example, the volumetric flow rate of the pump 280 may be designed to provide air at a particular velocity down the length 274 of the lumen 252, for example to provide turbulent flow along the interior of the lumen 252. The pump 280 enables a user to rapidly decontaminate the device 250 that may have a high length to width ratio, and may otherwise require a greater concentration of vaporized decontaminating substance 236 to perform a suitable decontamination process. The pump 280 thus allows a user to decontaminate the device 250 having a lumen 252 faster and with lower concentrations of decontaminating substance 236, thus allowing a user to avoid potential damage to the device 250.

The pump 280 may be powered by any suitable means, including battery and an external power source. Powering the pump 280 by battery allows the pump 280 to be placed anywhere within the container 260 without the need for a connection to a power source outside the container 260. The pump 280 may be powered by a power source external to the container, for example, by magnetic coupling the pump 280 to a power source located outside the container 260 which turns the pump 280 inside the container 260.

In some embodiments, the pump 280 may be placed in the container 260 and turned on before closing the door 230 to the decontamination chamber 220. In some embodiments, the pump 280 may be placed within the container 260 and the container 260 sealed with the pump 280 inside. In some embodiments, the pump 280 may be placed within the container 260, and the pump 280 turned on before the container 260 is sealed. Additionally or alternatively, the pump 280 may include a sensor that detects an air pressure inside the decontamination chamber 220 or inside the container 260. When a suitable or specified air pressure inside the decontamination chamber 220 or inside the container 260 is detected, the pump 280 may be configured to turn on.

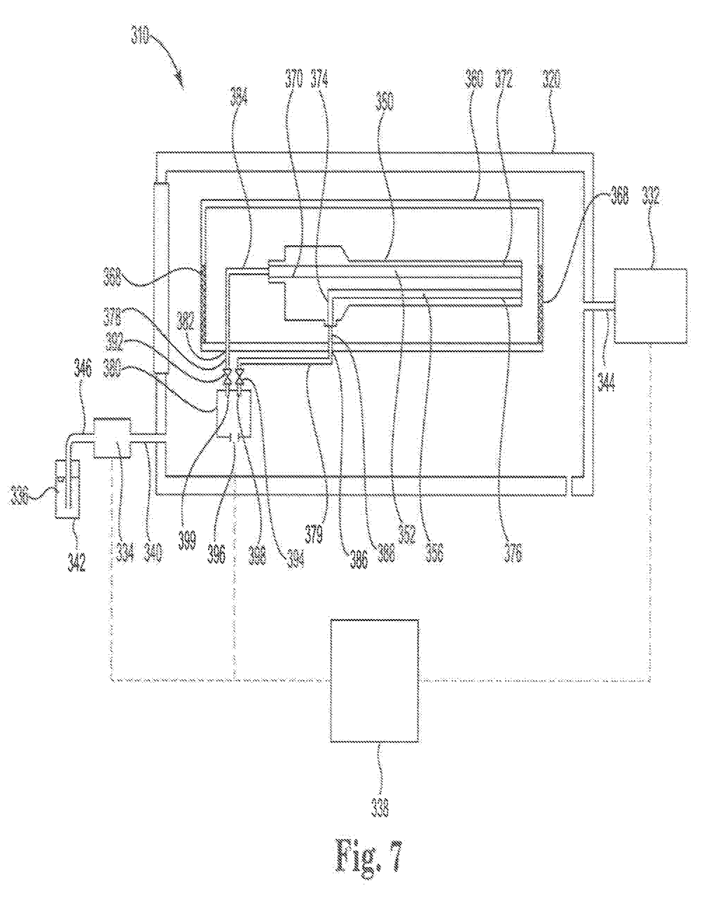

FIG. 7 is a schematic view of a decontamination system 310 for decontamination of a lumen device 350 that includes two lumens, namely first lumen 352 and second lumen 356. FIG. 7 shows similar features as previously described in reference to FIGS. 1 and 6, with comparable element numbers preceded with a 3.

As shown in FIG. 7, a decontamination system 310 includes a decontamination chamber 320, a vacuum pump 332, a vaporizer 334, a source of decontaminating substance 336 maintained in a package 342, a controller 338, a device 350 positioned within a container 360, and a pump 380. Vacuum pump 332 is connected to decontamination chamber 320 by conduit 344. Vaporizer 334 is connected to decontamination chamber 320 by conduit 340. Decontaminating substance 336 is maintained in package 342 and is connected to vaporizer 334 by conduit 346. Controller 338 can be connected to and configured to control one or more of the vacuum pump 332, vaporizer 334, and pump 380.

The container 360 can be similar to container 60 as described with respect to FIG. 1. Additionally, container 360 can include one or more ports, such as first port 382 and second port 386. In some embodiments, the first port 382 may include portion on the outside of the container 360 for connecting to conduit 378 and a portion on the inside of the container 360 for connecting to conduit 384. A membrane permeable by vaporized decontaminating substance 336 may be positioned between the portion of the first port 382. In this way, vaporized decontaminating substance 336 can permeate into the container 360 during a decontamination process and allows a sterile environment to be maintained within the container 360 after the container 360 is removed from decontamination chamber 320

The pump 380 is positioned within the decontamination chamber 320 and outside of the container 360. The pump 280 has an inlet 396, a first outlet 398 and a second outlet 399. Fluid enters the pump 380 through the inlet 396 and exits the pump 380 through the outlets 398 and 399.

The inlet 396 is exposed to, or is in fluid communication with, the environment within the decontamination chamber 320. The outlets 398 and 399 are connected to device 350 through conduits 378 and 379 so that pump 380 forces air from decontamination chamber 320 into first and second lumens 352, 356 of device 350. For example, during a decontamination cycle, the pump 380 directs air containing decontaminating substance 336 into the first and second lumens 352, 356 of the device 350. The pump 380 is configured to provide a sufficient flow rate so that the air from decontamination chamber 320 flows the entire length of the first and second lumens 352, 356.

In some embodiments, a first valve 392 may be included between the pump 380 and the first port 382, and a second valve 394 may be included between the pump 380 and the second port 386. The first and second valves 392, 394 regulate flow between the pump 380 and the first and second port, 382, 386. The first valve 392 may control a first flow rate of air containing vaporized decontaminating substance 336 from the pump 380 and the first lumen 352. The second valve 394 may control a second flow rate of air containing vaporized decontaminating substance 336 from the pump 380 and the second lumen 356.

The first and second valves 392, 394 may control the first and second flow rates independent of each other. In some embodiments, first and second valves 392, 394 may be controlled such that the decontaminating substance 336 flows through a single lumen at a time. For example, the first valve 392 may be closed when the second valve 394 is open and the decontaminating substance 336 can flow through the second lumen 356 while it is prevented from flowing through the first lumen 352.

In some embodiments, the device 350 may include first and second lumens 352, 356 that have different lengths or internal diameters. The first and second valves 392, 394 may be used to control a first and second flow rate independently to each of the first and second lumens 352, 356 that is suitable for the particular internal diameter of each of the first and second lumens 352, 356.

In some embodiments, the pump 380 may be configured to force air from within the decontamination chamber 320 into the device 350 to encourage decontamination of the first and second lumens 352, 356. For example, the pump 380 may draw air that contains vaporized decontaminating substance 336 from within the decontamination chamber 320 and send it through the first and second ports 382, 386 into the container 360. The device 350 may be connected to the first and second attachments 384, 388 on the inside of the container 360 at the first lumen 352 first end 370 or second end 372, and the second lumen 356 first end 374 or second end 376. The pump 380 may push air that contains vaporized decontaminating substance 336 from within the decontamination chamber 320 into the first end 370 of the first lumen 352, and/or the first end 374 of the second lumen 356.

In some embodiments, the pump 380 may be engaged to draw air from the first end 370 of the first lumen 352 and/or the first end 374 of the second lumen 356 which causes air that contains vaporized decontaminating substance 336 to be drawn into the second end 372 of the first lumen 352 and/or the second end 376 of the second lumen 356. By drawing air from the container 360 into the first and second lumens 352, 356, air in the decontamination chamber 320 that contains the decontaminating substance 336 may permeate a surface 368 of the container 360, and be drawn into the second end 372, 176 of the first and second lumens 352, 356.

The pump 380 may be configured to provide a volumetric flow rate that is suitable for the internal volume of the first and second lumens 352, 356. The volumetric flow rate of the pump 380 may provide air at a particular velocity down the length of each of the first and second lumens 352, 356. Pump 380 and valves 392, 394 may provide turbulent flow along the interior of the first and second lumens 352, 356. Depending on the interior diameter of each of the first and second lumens 352, 356, the pump 380 and/or valves 392, 394 may be adjusted to provide air at a volumetric flow rate that is laminar or turbulent, depending on the required configuration. The pump 380 may be designed to provide air at a volumetric flow rate to provide a particular amount of decontaminating substance 336 at a particular rate. For example, the concentration of vaporized decontaminating substance in the air in the decontamination chamber 320 may be calculated, and the required volume of air that contains a suitable amount of decontaminating substance 336 may be forced through the first and second lumens 352, 356 in a suitable amount of time to achieve a required decontamination.

In some embodiments, the pump 380 may attached to the second ends 372, 376 of the first and second lumens 352, 356. In some embodiments, the pump 380 may push air into, or pull air from, the second ends 372, 376 of the first and second lumens 352, 356. In some embodiments, there may be spacing between the first and second attachments 384, 388 on the inside of the container 360 and the first and second lumens 352, 356. That is, the first and second lumens 352, 356 may be positioned within the container 360 with a gap between the device 350 and the first and second attachments 384, 388 such that the first and second lumens 352, 356 and the first and second attachments 384, 388 do not touch each other. In some embodiments, there may be spacing between the first and second attachments 384, 388 and the device 350, and the first and second attachments 384, 388 may be shaped to direct air into the device 350. For example, the first and second attachments 384, 388 may be shaped with a tapered end to direct air into the first and second lumens 352, 356 of the device 350 without the first and second lumens 352, 356 touching the device 350. The first and second attachments 384, 388 may be positioned with a central axis of each of the first and second attachments 384, 388 in alignment with a central axis of the first and second lumens 352, 356 of the device 350 to direct a suitable flow of air into the device 350.

The pump 380 enables a user to rapidly decontaminate a device 350 that may have one or more lumens with a high length to width ratio that may otherwise require a greater concentration of vaporized decontaminating substance 336 to perform a suitable decontamination process. The pump 380 thus allows a user to decontaminate a device 350 having one or more lumens faster and with lower concentrations of decontaminating substance 336, thus allowing a user to avoid potential damage to the device 350. Additionally, a lower concentration of the decontaminating substance 336 provides a decontaminating process that requires less decontaminating substance 336 and less time to operate the decontamination system 310, thus decreasing the cost of the decontamination process.

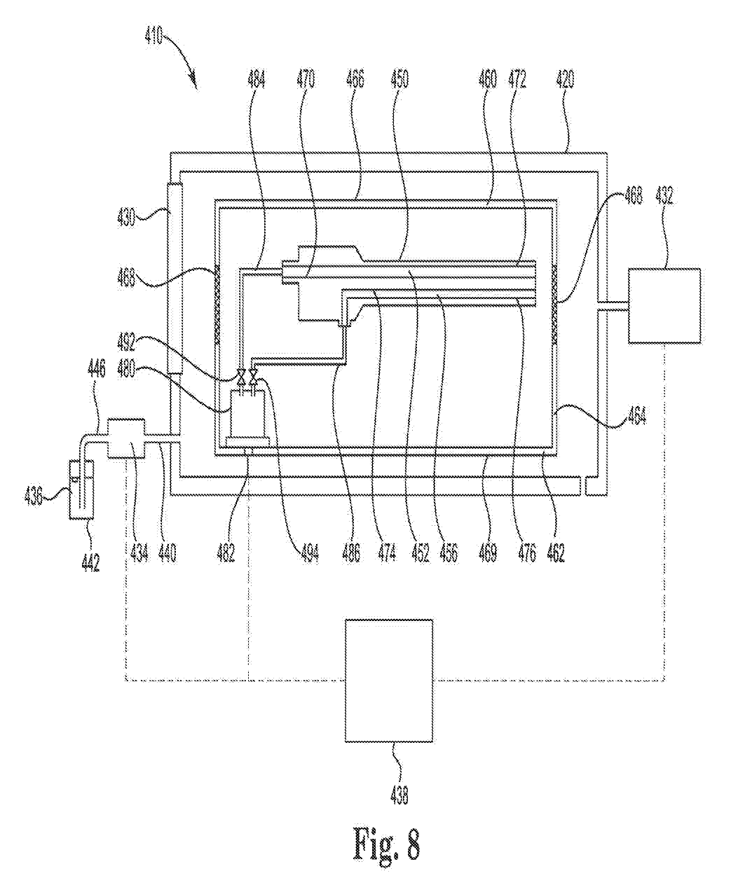

FIG. 8 is a schematic view of a decontamination system 410. The decontamination system 410 includes a decontamination chamber 420, a vacuum pump 432, a vaporizer 434, a source of a decontaminating substance 436, a controller 438, a conduit 440 and a package 442 for containing and/or delivering the decontaminating substance 436. Decontaminating substance 436 is maintained in package 442 and is connected to vaporizer 434 by conduit 446. The decontamination chamber 420 may have a door 430, configured to open and receive a device to be decontaminated 450 that is received within a container 460. The door 430 may be closed and sealed, and maintain a pressure inside the decontamination chamber 420 that is higher or lower than a pressure outside the decontamination chamber 420.

The decontamination system 410 shown in FIG. 8 may be used to decontaminate a first lumen 452 having a first end 470 and a second end 472, and a second lumen 456 having a first end 474 and a second end 476.

The container may have a bottom 462, a top 466, and a side portion 464. The container 460 may define a surface 469. The container 460 may have at least one portion, for example at least a first surface 468, through which the decontaminating substance 436 may penetrate or permeate. As shown in FIG. 7, the pump 480 may be included within the container 460. In some embodiments, the decontamination system 410 may include a port 482, for directing flow into the container 460 that contains the device to be decontaminated 450. In some embodiments, the port 482 may be constructed to provide fluid communication from outside the container 460 to within the container 460.

The pump 480 may be an air pump, a fan, a blower, compressor, or bellows for forcing air within the decontamination chamber 420. The pump 480 may be in fluid communication with the port 482. The container 460 may include a first and second attachment 484, 486 inside the container 460. The first attachment 484 may be connected to the first lumen 452 first or second end 470, 472; and the second attachment 486 may be connected to the second lumen 456 first or second end 474, 476. A first valve 492 may be included between the pump 480 and the first attachment 484, and a second valve 494 may be included between the pump 480 and the second attachment 486. The first and second valves 492, 494 may regulate flow between the pump 480 and the first and second attachments 484, 486 that are connected to the first and second lumens 452, 456. The first and second valves 492, 494 may be used to control a first and second volumetric flow rate independently to each of the first and second lumens 452, 456 that is suitable for the particular internal diameter of each of the first and second lumens 452, 456.

The pump 480 may draw air from within the decontamination chamber 420, through the port 482 and into the device 450 to provide decontamination of the lumens 452, 456. The pump 480 may be configured to draw air that contains vaporized decontaminating substance 436 from within the decontamination chamber 420 through the port 482 into the container 460. The pump 480 may force air that contains vaporized decontaminating substance 436 into the first and/or second attachment 484.486 on the inside of the container 460 and into the first and second lumens 452, 456. For example, the device to be decontaminated 450 may be connected to the first and/or second attachment 484, 486 on the inside of the container 460 at the first lumen 452 first end 470 or second end 472, and/or the second lumen 456 first end 474 or second end 476. The pump 480 may draw air that contains vaporized decontaminating substance 436 from within the decontamination chamber 420 through the port 482, and send it through the first attachment 484 and into the first end 470 of the first lumen 452. In some embodiments, the pump 480 may draw air out of the first end 470 of the first lumen 452 by creating a vacuum in the first end 470 of the first lumen 452 which causes air that contains vaporized decontaminating substance 436 to be drawn from within the container 460 into the second end 472 of the first lumen 452.

The pump 480 may be configured to provide a volumetric flow rate that is suitable for the internal volume of the first and second lumens 452, 456. The volumetric flow rate of the pump 480 may provide air at a particular velocity down the length of each of the first and second lumens 452, 456. Pump 480 and valves 492, 494 may provide turbulent flow along the interior of the first and second lumens 452, 456. Depending on the interior diameter of each of the first and second lumens 452, 456, the pump 480 and/or valves 492, 494 may be adjusted to provide air at a volumetric flow rate that is laminar or turbulent, depending on the required configuration. The pump 480 may be designed to provide air at a volumetric flow rate to provide a particular amount of decontaminating substance 436 at a particular rate. For example, the concentration of vaporized decontaminating substance 436 in the air in the decontamination chamber 420 may be determined, and the required volume of air that contains a suitable amount of decontaminating substance 436 may be forced through the first and second lumens 452., 456 in a suitable amount of time to achieve a required decontamination.

The pump 480 may be battery powered, which may allow it to be placed within the container 460 without the need for a connection to a power source outside the container 460. For example, the pump 480 may be placed into the container 460 and turned on before dosing the door 430 to the decontamination chamber 420. Alternatively, the pump 480 may include a sensor that detects an air pressure inside the decontamination chamber 420. When a suitable air pressure inside the decontamination chamber 420 is detected, the pump 480 may be configured to turn on. In some embodiments, the pump 480 may be powered by a magnetic coupling (not shown) to a power source located outside the decontamination chamber 420 which turns the pump 480 inside the decontamination chamber 420.

The pump 480 enables a user to rapidly decontaminate a device 450 that may have lumens with a high length to width ratio that may otherwise require a greater concentration of vaporized decontaminating substance 436 to perform a suitable decontamination process. The pump 480 thus allows a user to decontaminate a device 450 having lumens faster and with lower concentrations of decontaminating substance 436, thus allowing a user to avoid potential damage to the device 450. Additionally, a lower concentration of the decontaminating substance 436 provides a decontaminating process that requires less decontaminating substance 436 and less time to operate the decontamination system 410, thus decreasing the cost of the decontamination process.

It is desirable to reduce the time required for a decontamination cycle and process while still achieving the desired decontamination level. Decreasing the time required for effective decontamination of a device allows a user to decontaminate a larger number of devices in less time. Forcing decontaminating substance through a lumen of the device allows a user to directly flow decontaminating substance into a device that has an elongated and/or tortious flow path. For example, endoscopes or other devices that have lumens with a high length to width ratio may benefit from having the decontaminating substance directly injected into the interior of the lumen.

By directly injecting decontaminating substance into the interior of a lumen, a more effective means for the decontaminating substance to contact the interior surface of the lumen is provided, and may provide a decontaminating process that requires less decontaminating substance than a process that does not include a pump. This process also ensures that the entire interior surface of the lumen comes in contact with the decontaminating substance. That is, direct injection increases the ability for the decontaminating substance to penetrate the entire length of the lumen. One potential benefit of directly injecting decontaminating substance into a lumen is the decreased cycle time required for adequate decontamination along the entire length of the lumen.

Using the system and methods described above, it has been found that the decontamination system disclosed herein can effectively sterilize lumens 1.0, 2.0, 3.0 or 4.0 meters in length. The process disclosed herein has been found to successfully sterilize a lumen 3.5 meters in length, while maintaining the operating parameters of the decontamination cycle within the pressure and temperature tolerances of the lumen. The process disclosed herein has been found to successfully sterilize lumens with inner diameters of 1 mm, 1.6 mm, 2 mm, and 3.45 mm and an outer diameter of 3 mm, 3.18 mm, 4 mm, and 4.76 mm, and any value in between. It has also been found that as little as 2.0, 1.0, or 0.9 mL of decontaminating substance containing 59% hydrogen peroxide is successful in decontaminating multiple lumens simultaneously.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above described features.

What is claimed is:

1. A system for decontaminating a device, the system comprising:
a chamber defining an enclosed space, the chamber configured to withstand pressure changes within the enclosed space and receive a device that includes a lumen;
a controller configured to control a temperature and pressure within the chamber;
a vacuum pump in fluid communication with the enclosed space;
a source of a decontaminating substance;
at least one of a vaporizer or atomizer in fluid communication with the source of the decontaminating substance and configured to provide a vaporized decontaminating substance within the enclosed space; and
a pump positioned within the chamber, the pump having an inlet and an outlet wherein one of the inlet and outlet is in fluid communication with the enclosed space of the chamber and the other of the inlet and outlet is configured to be in fluid communication with the lumen.

2. (canceled)

3. The system of claim 1, wherein the inlet of the pump is in fluid communication with the enclosed space and the outlet of the pump is configured to be in fluid communication with the lumen.

4. The system of claims 1, wherein the outlet of the pump is in fluid communication with the enclosed space and the inlet of the pump is configured to be in fluid communication with the lumen.

5. The system of claim 1, wherein at least one of the inlet or outlet of the pump is configured to be in fluid communication with the lumen through a conduit attached to the pump and the lumen.

6. The system of claim 1, wherein the outlet of the pump is configured to be in fluid communication with the lumen through a conduit that is spaced apart from the device and is shaped to direct flow from the outlet of the pump through the lumen.

7. The system of claim 1, further comprising a container positioned within the enclosed space and enclosing the device, the container formed by at least one outer wall that is permeable by the vaporized decontaminating substance, wherein the pump is positioned outside the container to provide a flow of the vaporized decontaminating substance through the outer wall of the container to the lumen.

8. The system of claim 1, further comprising a container positioned within the enclosed space and enclosing the device, the container formed by at least one outer wall that is permeable by the vaporized decontaminating substance, wherein the pump is positioned inside the container to provide a flow of the vaporized decontaminating substance to the lumen.

9. The system of claim 1, further comprising a conduit connected to the outlet of the pump for directing the decontaminating substance from the outlet of the pump to the lumen.

10. The system of claim 1, and further comprising a conduit having a first end connected to the pump and a second end in fluid communication with the lumen for directing the vaporized decontaminating to the lumen and an outer surface of the device.

11. The system of claim 1, wherein the decontaminating substance comprises peracetic acid.

12. A system for decontaminating a device having a lumen, the system comprising:
a decontamination chamber defining an enclosed space, the decontamination chamber configured to withstand pressure changes within the enclosed space;
a container positioned within the enclosed space, the container having an outer wall that is permeable by a vaporized decontaminating substance;
a controller for controlling a temperature and pressure within the decontamination chamber;
a vacuum pump in fluid communication with the enclosed space;
a source of a decontaminating substance;
at least one of a vaporizer or atomizer for vaporizing the decontaminating substance, the vaporizer or atomizer in fluid communication with the source of the decontaminating substance and the enclosed space; and
a pump positioned within the decontamination chamber, the pump having a first opening in fluid communication with the enclosed space and a second opening in fluid communication with the lumen.

13. (canceled)

14. The system of claim 12, wherein the device includes a plurality of lumens, and wherein the system further comprises at least a first valve configured for controlling fluid communication between the pump and the plurality of lumens.

15. The system of claim 12, wherein the pump is positioned within the container.

16. The system of any of claims 12 through 15, further comprising a conduit having a first end connected to the second opening of the pump and a second end configured to be connected to the lumen such that the lumen is directly connected to the second opening of the pump via the conduit.

17. The system of claim 12, further comprising a conduit connected to the second opening of the pump, the conduit spaced from the device and configured to direct a flow of air to the device without touching the device.

18. The system of claim 12, further comprising a conduit having a first end connected to the pump and a second end positioned to direct the vaporized decontaminating substance to the lumen and an outside surface of the device.

19. The system of claim 12, further comprising a conduit connected to the second opening of the pump, the conduit having an outer diameter smaller than an inner diameter of the lumen such that the conduit is configured to be positioned with the outer diameter of the conduit within the inner diameter of the lumen to direct a flow of air into the lumen without touching the device.

20. A method of decontaminating a device containing a lumen, the method comprising:
placing the device inside a container;
placing the container within a decontamination chamber, the decontamination chamber defining an enclosed space;
decreasing the pressure inside the enclosed space to a pressure below atmospheric pressure;
directing a vaporized or atomized decontaminating substance into the enclosed space; and
providing a flow of air containing the vaporized or atomized decontaminating substance from the enclosed space into a lumen of the device with a pump located within the enclosed space or the container.

21. The method of claim 20, and further including physically connecting the pump to the lumen.

22. (canceled)

23. (canceled)

24. The method of claim 20, wherein the decontaminating substance includes peracetic acid.
